(19) United States
(12) Patent Application Publication
(10) Pub. No.: US 2007/0190663 A1
(43) Pub. Date: Aug. 16, 2007

(54) System and method for using polarized or hyperpolarized contrast agent to perform parallel magnetic resonance imaging of a sample

(76) Inventors: Ray F. Lee, New York, NY (US); Georgeann McGunness, New York, NY (US); Glyn Johnson, New York, NY (US); Qun Chen, New Hyde Park, NY (US); Robert Grossman, New York, NY (US)

Correspondence Address: DORSEY & WHITNEY LLP;INTELLECTUAL PROPERTY DEPARTMENT, 250 PARK AVENUE, NEW YORK, NY, 10177, US

(21) Appl. No.: 11/504,570

(22) Filed: Aug. 14, 2006

Related U.S. Application Data
(60) Provisional application No. 60/707,679, filed on Aug. 12, 2005.

(51) Int. Cl. G01N 24/00; A61B 5/05

(52) U.S. Cl. 436/173; 600/410

(57) ABSTRACT

A system and method for performing parallel magnetic resonance imaging (“MRI”) with a polarizable or hyperpolarizable contrast agent are provided. The contrast agent may be provided in the form of a gas, a liquid, or a solid. The system may include a phased-array rigid coil with, e.g., a 24-channel phased-array for a receiver and a 2-channel large loop for a transmitter. The phased-array rigid coil can be connected to a low impedance preamplifier for decoupling the strong coupling between two adjacent coils in the phased-array rigid coil. The system and method may be used for enhancing the signal-to-noise ratio, and/or reducing the scan acquisition time of acquired images in a parallel MRI scan of a patient who is breathing a hyperpolarized gas.

### CROSS-REFERENCE TO RELATED APPLICATION(S)

This application claims the benefit of priority from U.S. patent application Ser. No. 60/707,679, filed Aug. 12, 2005, the entire disclosure of which is incorporated herein by reference.

### FIELD OF THE INVENTION

The present invention relates generally to magnetic resonance imaging (“MRI”). More specifically, the present invention relates to systems and methods for performing parallel MRI of a sample using hyperpolarized gases.

### BACKGROUND INFORMATION

MRI has emerged as a leading medical imaging technology for the detection and assessment of many pathological and physiological alterations in living tissue, including many types of tumors, injuries, brain-related conditions, coronary conditions, and orthopedic conditions, among others. One of the main advantages of this technique is that, according to current medical knowledge, an MRI scan of a patient is non-invasive and harmless to such patient. Other advantages of MRI techniques may include a high spatial resolution, superior anatomical imaging detail of soft tissues as compared to other medical imaging technologies, and an ability to acquire images in any plane.

An MRI scan generally utilizes magnetic and radio frequency (“RF”) fields to elicit a response from a given patient's tissue and to provide high quality image “slices,” i.e., two-dimensional image reconstructions of a two-dimensional cross-section of the patient's body, e.g., a tissue along with detailed metabolic and anatomical information. Radio waves 10,000-30,000 times stronger than the magnetic field of the earth are transmitted through the patient's body. This affects the patient's atoms by forcing the nuclei of some atoms into a different position. As such nuclei move back into place, they transmit their own radio waves. An MRI scanner picks up those radio waves, and a computer transforms them into images, based on the location and strength of the incoming magnetic waves.

Conventional MRI systems typically use disturbance of water protons to acquire images of the patient's tissue. A natural abundance of water in the body together with a large magnetic moment of water protons make them a preferred choice for most imaging applications. Water protons, however, may be difficult to image in certain biological environments of interest, e.g., the lungs and lipid bilayer membranes such as those in the brain. Other atoms are either present in very low concentrations and/or have undesirable magnetic resonance characteristics, thus limiting their use in obtaining MRI scans of these regions.

Certain difficulties encountered during the performance of MRI scans of a patient's lungs may be overcome, e.g., by increasing the polarization of gases they contain. In principle, this can be accomplished by strengthening the applied magnetic field.

Another approach has been developed for imaging lung tissue using MRI techniques. In this approach, the patient is requested to inhale a gas that has been hyperpolarized outside of the patient's body, and MRI scans may be acquired before the gas becomes depolarized. In this hyperpolarized MRI technique, as described for example in U.S. Pat. No. 5,545,396, the hyperpolarized gases may be ³He or ¹²⁹Xe. Because these gases are chemically inert, they are unlikely to produce any long-term adverse effects after inhalation, even upon repeated exposure. In addition to safety considerations, these gases may be advantageous because they lack molecular rotation. It has been observed that a nucleus in a rotating molecule has a much greater tendency to lose its polarization before a useful image can be acquired.

In particular, a hyperpolarized ³He gas MRI may be useful for accessing pulmonary ventilation, microstructural changes and gas exchange properties of the lungs. ³He has also been observed to produce higher quality images in MRI applications than 29Xe, it is cheaper to produce than ¹²⁹Xe, and the technology for polarizing ³He is more mature than the technology for polarizing ¹²⁹Xe.

Typically, for this technique, patients inhale ³He, and retain it in their lungs for about 20 seconds for the MRI scan to be performed. It may be preferable to reduce the time expended to acquire the MRI scans while maintaining, or even enhancing, the signal-to-noise ratio (“SNR”) of the acquired scans. Conventional approaches for improving the data acquisition process during hyperpolarized ³He lung MRI have used either birdcage type rigid quadrature coils or flexible wrap-around-chest quadrature coils, which only allow for sequential data acquisition. Recently, the potential of performing hyperpolarized ³He lung MRI scans in parallel has been reviewed.

For example, one study has demonstrated the feasibility of performing parallel MRI with hyperpolarized ³He by using a sequence which enables a number of slices to be encoded simultaneously with a flexible twin saddle quadrature transmit-receive coil. (see, e.g., http://www.shef.ac.uk/dcss/medical/radiology/research/chestimg/psd1.html). Parallel imaging techniques applied to hyperpolarized ³He may possibly be used to perform lung MRI procedures with a several-fold decrease in acquisition time and without a substantial adverse SNR effect as described in J. P. Mugler, III, and J. R. Brookeman, “Signal-to-Noise Considerations for Parallel Imaging with Hyperpolarized Gases,” Proc. Intl. Soc. Mag. Reson. Med. 13 (2005).

Thus, there may be a need to improved hyperpolarized ³He parallel MRI to image a patient's lungs.

There is a further need to provide a system and method for performing hyperpolarized ³He parallel MRI that allows for an improved scan acquisition time and an improved quality of the acquired images.

### SUMMARY OF THE INVENTION

In view of the foregoing, one of the objects of the present invention is to provide a system and method for performing hyperpolarized ³He parallel MRI to image a patient's lungs which addresses the above-described deficiencies.

It is another object of the present invention to provide a system and method for performing hyperpolarized ³He parallel MRI to improve the scan acquisition time and the quality of the acquired images.

A further object of the present invention is to provide a system and method for performing parallel MRI to image a patient's lungs or other parts of the body using hyperpolarized materials that may have a form of a liquid or a solid. The hyperpolarized liquid may be vaporized and inhaled, and/or ingested or injected into the body. A solid hyperpolarized material may be provided in a particulate or powder form and inhaled, and/or it may be ingested.

These and other objects of the present invention can be accomplished using an exemplary embodiment which can include a coil system (e.g., a rigid coil system) which includes a 24-channel phased-array for the receiver and a 2-channel large loop for the transmitter. The exemplary system can be used for enhancing the signal-to-noise ratio (“SNR”), and/or reducing the scan acquisition time of the acquired images in an MRI scan of a patient with hyperpolarized ³He. The coil system can include various numbers of elements such as, e.g., 128 elements or 256 elements. The coil system may be provided in various configurations such as, for example, a transmit/receive array, a receive only array, a loop array, a strip array, or any suitable phase array configuration. For example, elliptical, cylindrical or planar strip arrays may be used.

Because hyperpolarized gas relaxes rapidly and may not be reusable, parallel MRI (which may be accomplished by simultaneously acquiring multiple channel signals for spatial encoding using a phased array) can significantly reduce the scan acquisition time of the acquired images. A rigid coil with a phased-array can also be used to provide a higher SNR than the SNR produced by quadrature coils during a sequential data acquisition.

Advantageously, because the polarization within lungs generally decays rapidly, the SNR of the acquired images can be enhanced by exemplary embodiment of systems in accordance with the present invention. In addition, e.g., an eight fold (2×4) scan time reduction can be achieved without aliasing in images acquired via a three- dimensional data acquisition using an exemplary 1.5 T 24-ch ³He phased-array system.

### BRIEF DESCRIPTION OF THE DRAWINGS

The foregoing and other objects of the present invention will be apparent upon consideration of the following detailed description, taken in conjunction with the accompanying drawings and claims, in which like reference characters refer to like parts throughout, and in which:

FIG. 1 is a visual illustration of an exemplary embodiment of a system (e.g., a rigid coil apparatus) according to the present invention;

FIG. 2 is a visual diagram of an exemplary layout of the system according to the present invention which can include twenty four receive copper loops;

FIGS. 3A-B are visual illustrations of exemplary layouts of the two transmit rectangular loops and the twenty four receive coils provided in top and bottom shells in accordance with an exemplary embodiment of the present invention;

FIG. 4 is a schematic diagram of an equivalent circuit for modeling two adjacent coils when a low impedance preamplifier is used to decouple the strong coupling between adjacent loop pairs in the rigid coil in accordance with an exemplary embodiment of the present invention;

FIG. 5 is a an exemplary graph of impedance versus frequency of the exemplary equivalent circuit shown in FIG. 4 when no coupling between two adjacent coils is present;

FIGS. 6A-B are exemplary graphs of impedance versus frequency and inductance, and inductance versus frequency, when an exemplary coil pair is not connected to the exemplary equivalent circuit of FIG. 4;

FIG. 7 is a magnified view of the exemplary graph of inductance versus frequency of the equivalent circuit shown in FIG. 4 when the coupling between two adjacent coils is strong;

FIGS. 8A-D are magnified views of the exemplary graphs of inductance versus frequency of the equivalent circuit shown in FIG. 4 illustrating its performance under various operating conditions;

FIG. 9 is a schematic diagram of an exemplary embodiment of an external frequency synthesis system that may be used in a proton frequency system adapted to operate with ³He frequency coils and preamplifiers according to the principles of the present invention;

FIGS. 10A-B are exemplary graphs illustrating the impedance and the Q factor of the preamplifier when tuned to an exemplary resonance frequency of 48.5 MHz in accordance with an exemplary embodiment of the present invention;

FIGS. 11A-B are exemplary graphs illustrating the frequency splitting and frequency shifting effects of the preamplifier designed according to the exemplary embodiments of the present invention;

FIGS. 12A-F are exemplary graphs illustrating the S-parameters of twenty four units of 1T proton preamplifiers according to the exemplary embodiments of the present invention;

FIGS. 13A-B are exemplary graphs illustrating the noise figures and gains of the preamplifiers built according to the principles of the present invention; and

FIGS. 14A-C are exemplary images acquired using an exemplary embodiment of a phased-array coil according to the present invention.

Throughout the figures, the same reference numerals and characters, unless otherwise stated, are used to denote like features, elements, components or portions of the illustrated embodiments. Moreover, while the present invention will now be described in detail with reference to the figures, it is done so in connection with the illustrative embodiments.

### DETAILED DESCRIPTION OF EMBODIMENTS OF THE INVENTION

In accordance with certain exemplary embodiments of the present invention, a system is provided for performing parallel magnetic resonance imaging (“MRI”) with a gas (e.g., a polarized gas) such as hyperpolarized ³He, using a phased-array rigid coil. As stated herein, hyperpolarized ³He refers to ³He that has been hyperpolarized outside of a patient's body. Parallel MRI generally refers to an MRI scan of a patient in which more than one image is acquired simultaneously. A parallel MRI scan of a patient using hyperpolarized gases generally refers to a parallel MRI scan in which the patient breathes in the hyperpolarized gas into the patient's lungs, and the images are acquired before the gas becomes depolarized. A parallel MRI scan using hyperpolarized gases may be used to image the lungs of a patient, lipid bilayer membranes such as those in the brain, or other biological tissues.

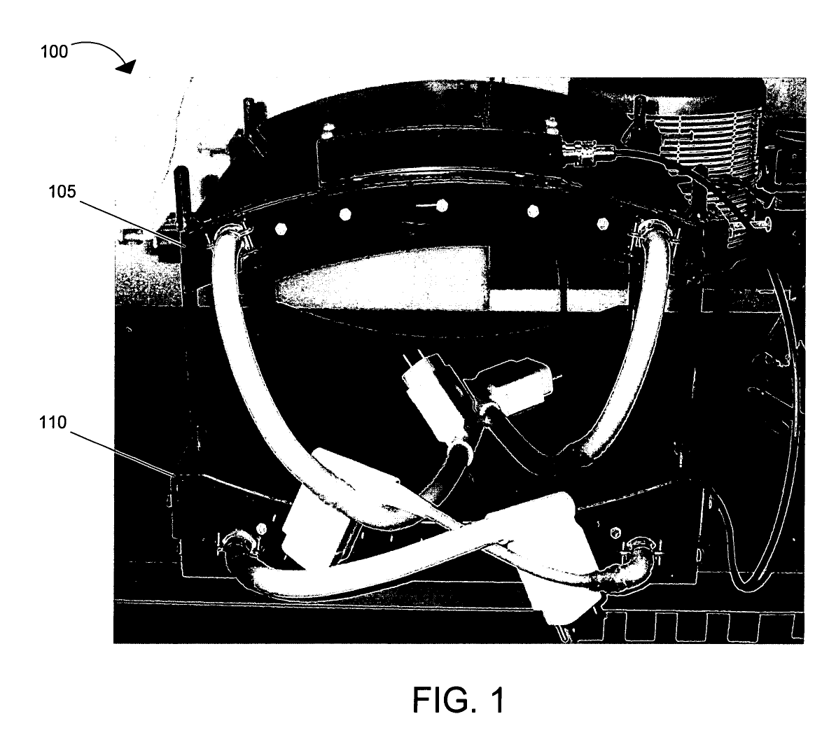

Referring to FIG. 1, a visual illustration of an exemplary embodiment of a rigid coil system 100 according to the present invention is provided. A rigid coil 100 has transmit and receive coils that are situated in one or more housings, where the housings may include two rigid shells, e.g., shells 105, 110. For example, two levels can be provided within each shell. The receive arrays can be within an inner level of each shell, and the transmit coils can be within an outer level of each of the shells 105, 110.

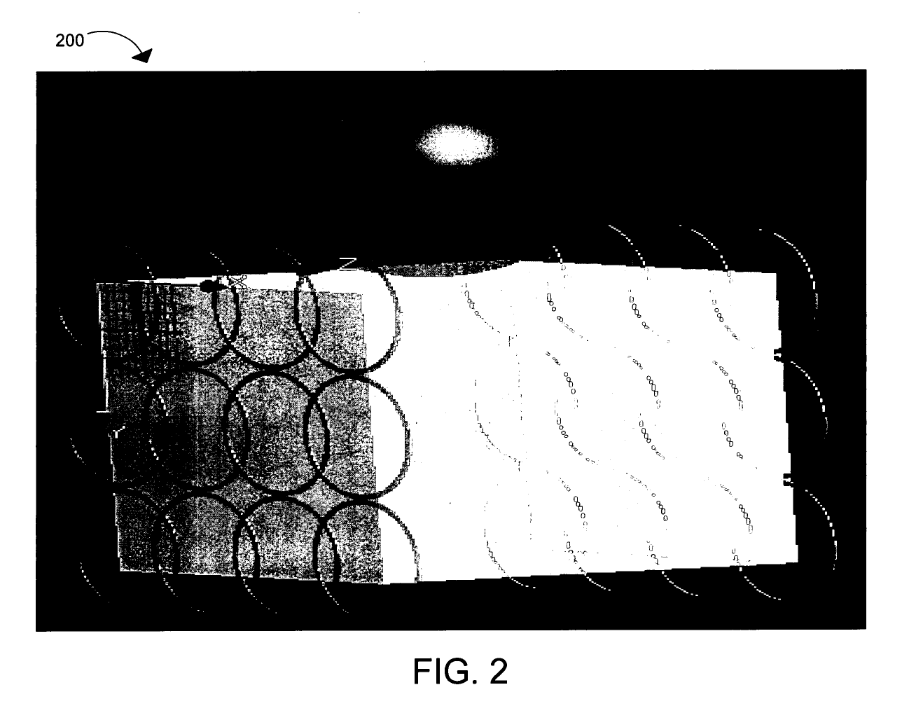

Referring to FIG. 2, an exemplary layout 200 of twenty-four receive copper loops of an exemplary embodiment of a system according to the present invention is provided. The exemplary layout 200 can be designed so as to provide a compromise between the competing requirements of decoupling of the coils and parallel imaging. In a left-to-right direction, overlapping of a nearest neighbor may reduce or eliminate a strong coupling between adjacent loop pairs. Weak coupling between non-adjacent pairs can be removed using low-impedance preamplifiers, as described in further detail herein below.

In order to avoid the complication of overlapping and parallel imaging encoding along a B0 direction, no overlapping in the superior-inferior direction may be applied. The layout 200 of FIG. 2 can be used with a low-impedance preamplifier to decouple the strong coupling between adjacent loop pairs. The availability and potential limitations of such an amplifier are discussed in more detail herein below.

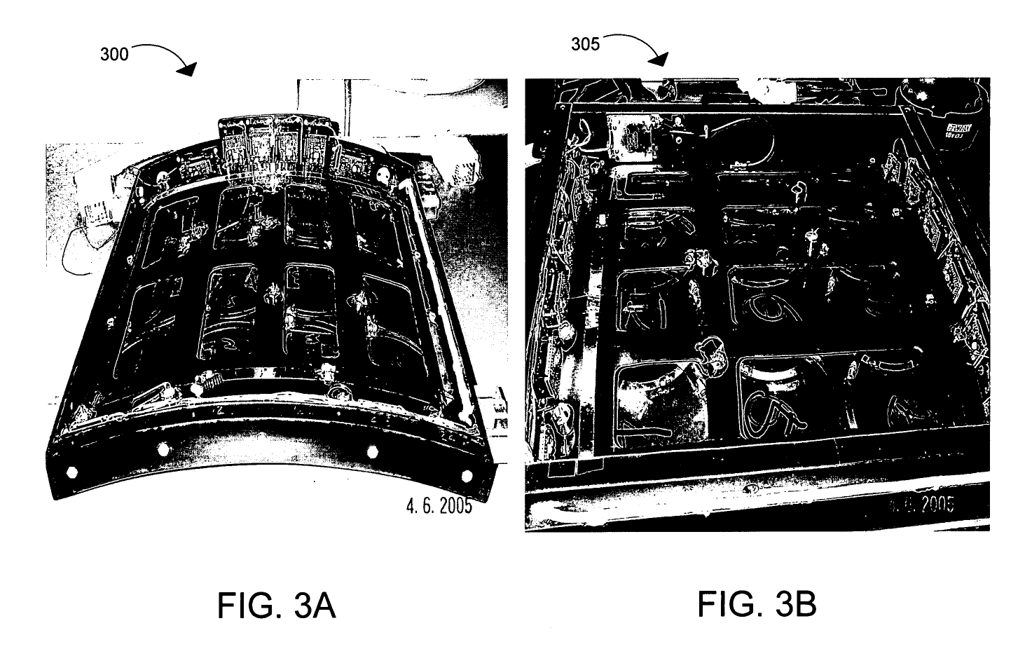

Referring to FIGS. 3A-B, a visual illustration is provided of exemplary configurations of two transmit rectangular loops and twenty four receive coils in top and bottom shells in accordance with an exemplary embodiment of the present invention. For example, the transmit coils (provided in a configuration shown in illustration 300 in FIG. 3A) can have dimensions of approximately 40 cm×37 cm. The receive coils may be provided in a configuration such as that shown in illustration 305 in FIG. 3B.

The quantitative effects of using a low-impedance preamplifier to decouple the strong coupling between adjacent loop pairs are described, e.g., in R. F. Lee et al., “Coupling and decoupling theory and its applications to the MRI phased-array,” Magnetic Resonance in Medicine, vol. 48, pp. 203-213, 2002, incorporated herein by reference in its entirety. This publication describes modeling of any two adjacent coils using a corresponding equivalent circuit.

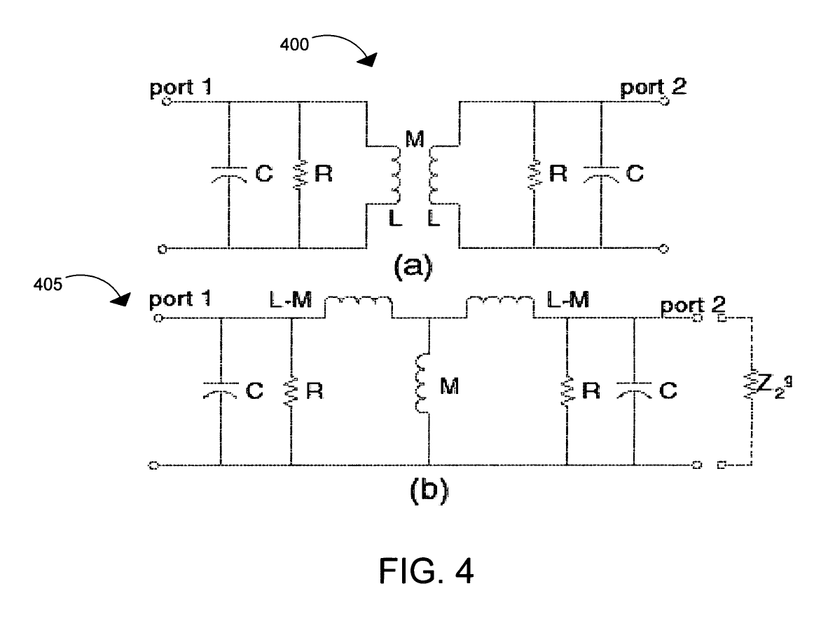

Referring to FIGS. 4A-B, schematic diagrams of two equivalent circuits are provided. These circuits 400, 405 may be used for modeling two adjacent coils when a low impedance preamplifier is present, in accordance with an exemplary embodiment of the present invention. Such configuration can be used to decouple strong coupling between adjacent loop pairs in a rigid coil. In the exemplary equivalent circuits 400, 405, L can represent the inductance of a loop, M can represent the mutual inductance between two loops, C can represent a tuning capacitor, R can represent a shunted loading resistance, and Z_{g} can represent the impedance of the preamplifier.

As described in the R. F. Lee publication, the impedance can be measured at one port, while another port may be terminated with a low impedance of the preamplifier, which can be expressed as:

$${Z}_{1} = {Z}_{11} - \frac{{Z}_{12}^{2}}{{Z}_{22} + {Z}_{2}^{g}} = {Z}_{11} - \frac{{Z}_{12}^{2}}{{Z}_{11} + {Z}_{1}^{g}} = \frac{a + j b}{c + j d} \qquad ( 1 )$$

where ω is the angular frequency, and
a=−ω²(L²−M²)(R²+RZ₁^{g}); (2)
b=ωLR²Z₁^{g}−ω³CR²Z₁^{g}(L²−M²); (3)
c=R²Z₁^{g}−ω²((L²−M²)(R+Z₁^{g})+2CLR²Z₁^{g})+ω⁴C²R²Z₁^{g}(L²−M²); and (4)
d=ωLR(R+2Z₁^{g})−ω³(L²−M²)CR(R+2Z₁^{g}) (5)

In an exemplary model, parameters of the circuits 400, 405 can be set to, e.g., L=280 nH, C=38.45 pF, and R=10KΩ, where these approximate values may be based on experimental observations.

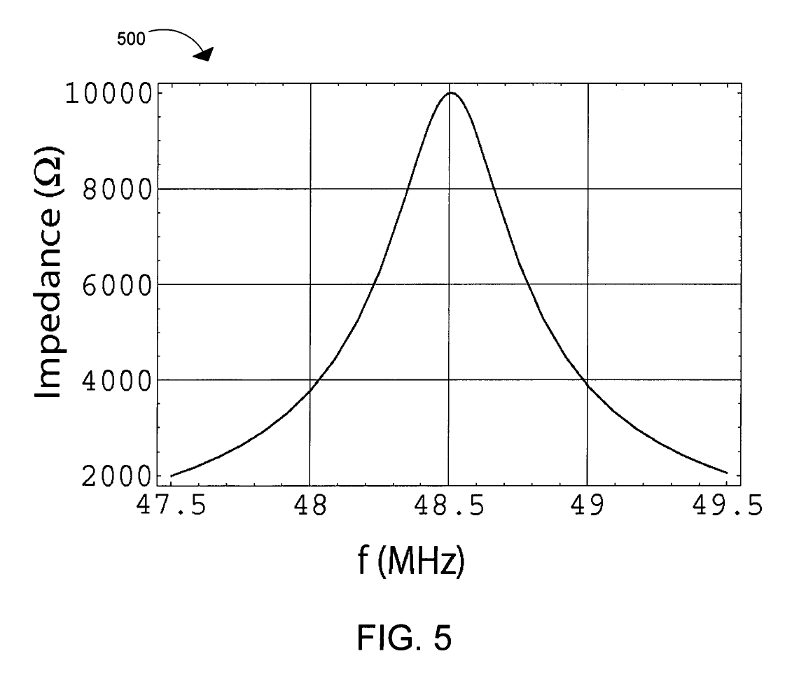

Referring to FIG. 5, an exemplary graph 500 of impedance versus frequency for the exemplary preamplifier shown in FIG. 4 (when no coupling is present between two adjacent coils) is provided. The exemplary graph 500 indicates that when no coupling is present between two adjacent coils in the equivalent circuit of FIG. 4 (e.g., when the mutual inductance M between two loops is zero), each coil may be tuned to the ³He resonance frequency of 48.5 MHz. If another hyperpolarizable material is used, each coil may be tuned to an appropriate resonance frequency of that material.

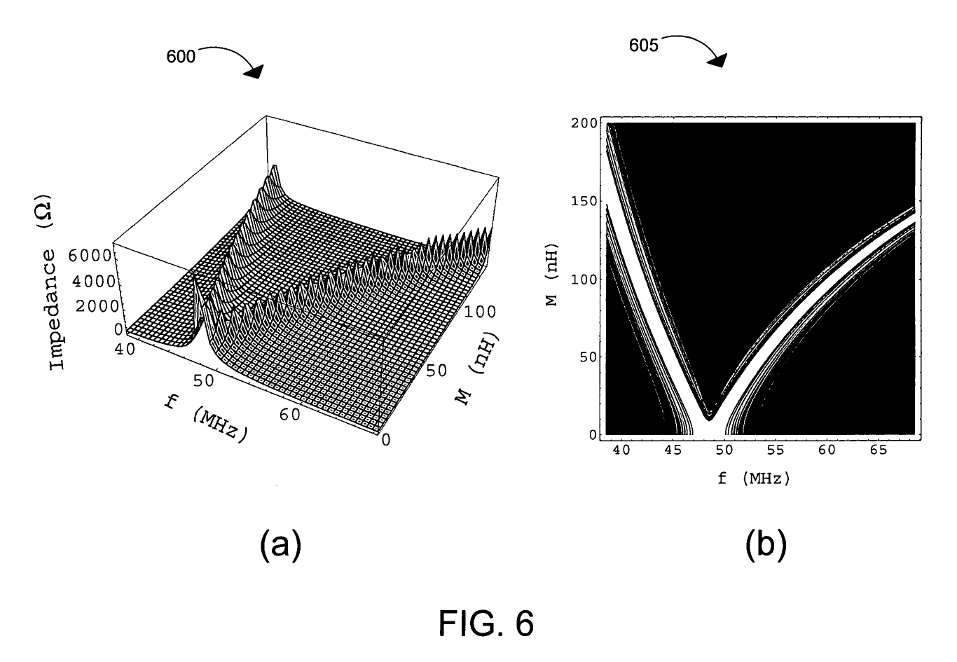

Referring to FIGS. 6A and 6B, an exemplary graph 600 of impedance versus frequency and inductance, and an exemplary graph 605 of inductance versus frequency are provided. These exemplary graphs 600, 605 correspond to a configuration where a coil pair is not connected to the equivalent circuits of FIG. 4. The exemplary graph 600 is a three-dimensional graph showing the impedance L of the loop versus the mutual inductance M between two loops and the resonance frequency of the equivalent circuits of FIG. 4. The exemplary graph 605 is a graph of the mutual inductance M between two loops and the resonance frequency of the equivalent circuit of FIG. 4. As illustrated in the graphs 600, 605, when the coil pair is not connected with the preamplifier (e.g., when Z_{g}=10KΩ and an open circuit is effectively formed), the mutual inductive coupling of two coils may increase. As a result, the resonance frequency of the exemplary equivalent circuit of FIG. 4 can begin to split and shift away from the ³He resonance frequency of 48.5 MHz.

However, if the coils are connected with an ideal low impedance preamplifier, e.g., when Z_{g}=0, no frequency splitting occurs. In the weak coupling case, e.g., when M<20% L, the resonance frequency of two coils likely remains the same. When the coupling becomes strong, such non-splitting frequency may shift to a higher frequency.

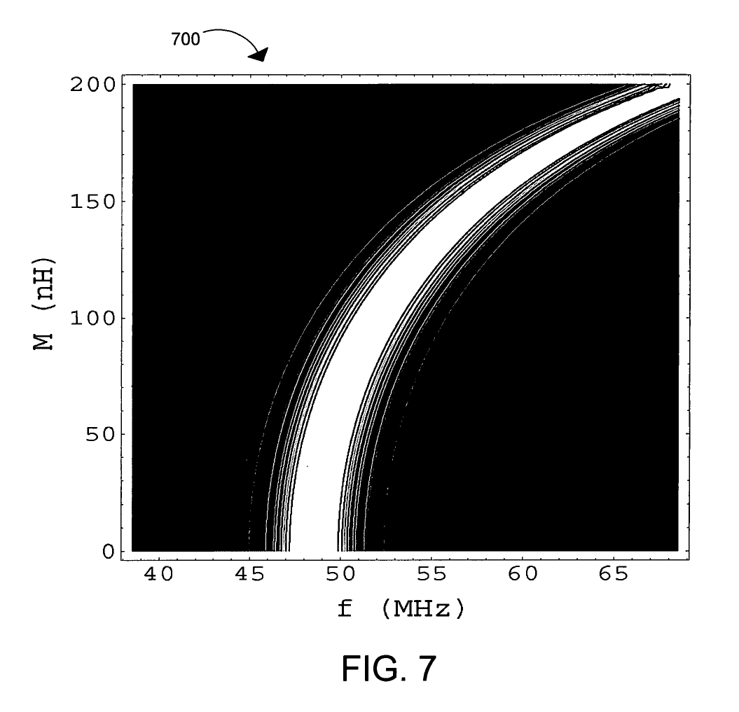

Referring now to FIG. 7, a magnified view of an exemplary graph 700 of inductance versus frequency of the equivalent circuit (shown in FIG. 4) when the coupling between two adjacent coils is strong is provided. The exemplary graph 700 indicates a presence of a non-splitting frequency shift to a higher frequency when the coupling between two adjacent coils is strong. This additional frequency shift can complicate tuning of strongly coupled coil pairs if only preamplifiers are used to decouple them.

Generally, a preamplifier may not maintain its stability when its input impedance Z_{g} is zero. The exemplary model according to the present invention, provided in Equations (1)-(5) hereinabove, indicates that if Z_{g} is about 2Ω (which can be achieved in some low-noise preamplifiers), the decoupling capability of the preamplifier can be maintained.

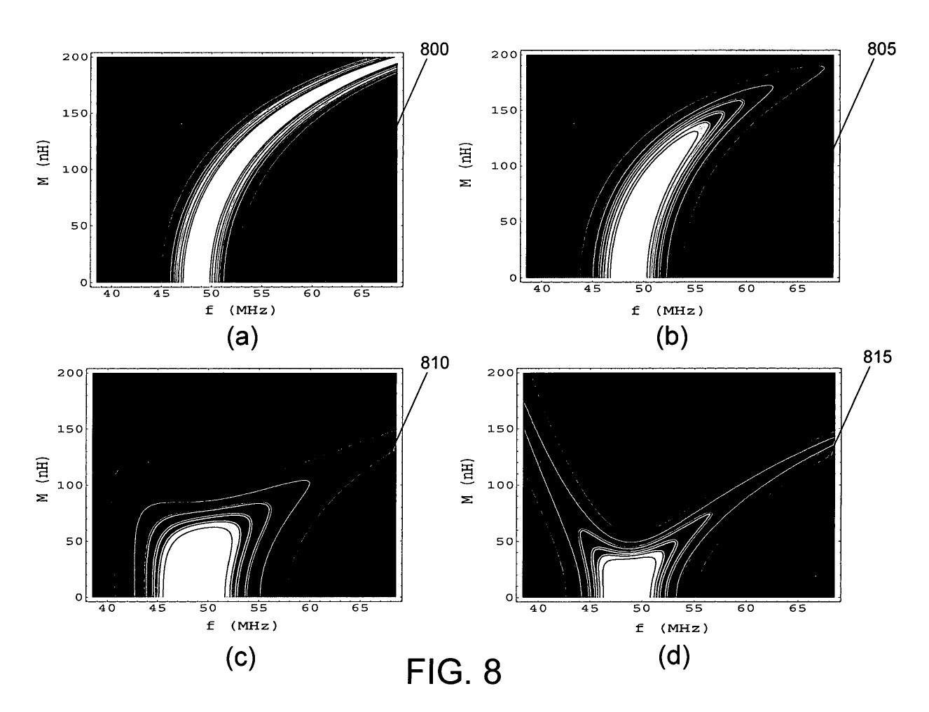

Referring now to FIGS. 8A and 8D, magnified views are provided of exemplary graphs 800, 805, 810, 815 of inductance versus frequency of the equivalent circuit shown in FIG. 4 illustrating its performance under various operating conditions. The decoupling capability of the preamplifier when Z_{g} is about 2Ω is provided in the graph 800 in FIG. 8A. The exemplary graph 805 in FIG. 8B provides that when Z_{g} is approximately 20Ω, the preamplifier can still avoid frequency splitting in both the weak and strong coupling cases. The Q-factor of the coil may be significantly reduced at this impedance level, especially if coupling is strong. When Z_{g} is approximately 200Ω, the decoupling function of the equivalent circuit of FIG. 4 may be reduced or even disappear when the coupling is strong, as shown in the graph 810 of FIG. 8C. When Z_{g} is approximately 500Ω, the preamplifier may not even decouple the weak coupling, as shown in the graph 815 of FIG. 8D.

The graphs 800-815 shown in FIG. 8 can quantitatively indicate that to use a preamplifier to decouple non-overlapped strongly-coupled coil pairs (similar to the layout in superior-inferior direction shown in FIGS. 3A-B), the input impedance of the preamplifier may be approximately 2Ω. To enable a proton frequency system to operate with ³He frequency coils and a preamplifier according to exemplary embodiments of the present invention, external frequency synthesis circuitry may be used.

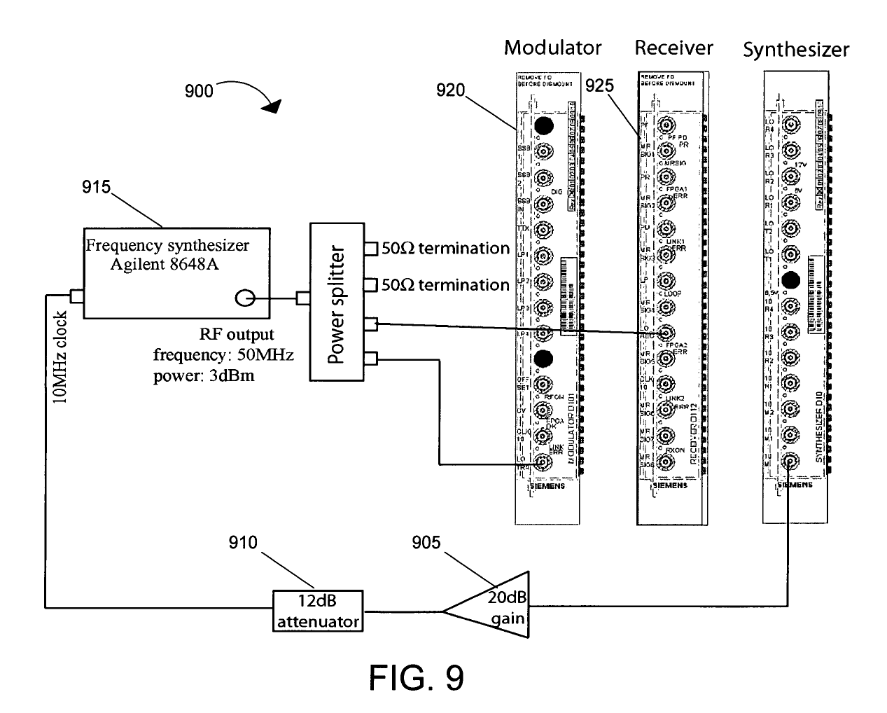

FIG. 9 shows a schematic diagram of an exemplary embodiment of an external frequency synthesis circuitry used in a proton frequency system adapted to operate with ³He frequency coils and preamplifiers according to the principles of the present invention. The exemplary external frequency synthesis circuitry 900 may include, e.g., a 10 MHz clock for a synchronization between an external exemplary frequency mixing system and an exemplary MRI scanner. Such 0.3 dB clock can be amplified to, e.g., 20 dB with amplifier 905, and attenuated to 12 dB with attenuator 910. The exemplary clock can then be provided to a frequency synthesizer 915, such as the Agilent 8648A, sold by Agilent Technologies Inc., of Palo Alto, Calif. A 50 MHz output of the frequency synthesizer 915 may be provided to a modulator board 920 and a receive board 925 of the MRI scanner so as to change their respective local oscillation frequencies to transmit and receive 48.5 MHz ³He physiological signals.

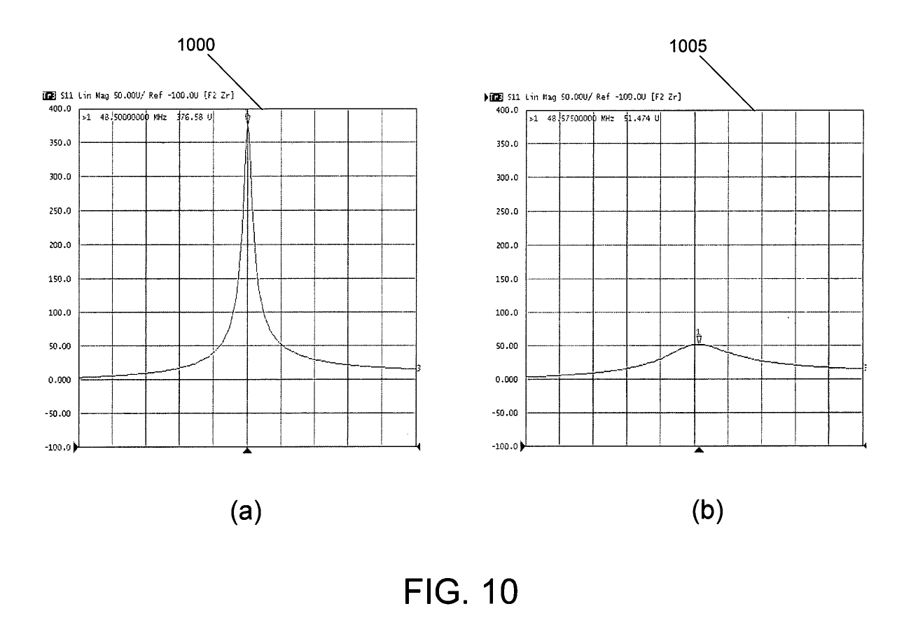

Certain measurements can be performed using the external frequency synthesis circuitry 900 of FIG. 9 by tuning each of the 24 loop coils to 48.5 MHz. Referring to FIGS. 10A and 10B, exemplary graphs 1000, 1005 are provided which indicate the impedance and the Q factor of the preamplifier when tuned to an exemplary resonance frequency of 48.5 MHz. The unloaded impedance of the external frequency synthesis circuitry 900 of FIG. 9 can be set to be about 370Ω, and its unloaded Q-factor may be about 405. When the coil is loaded, its impedance can be matched to 50Ω, and its Q may be 49, as shown in the graphs 1000 and 1005 in FIG. 10, respectively.

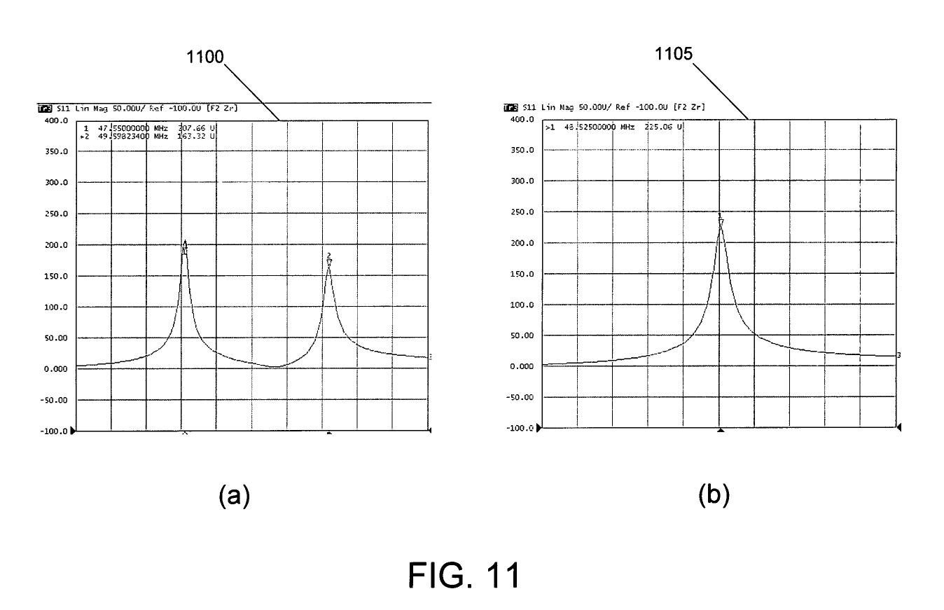

Referring to FIGS. 11A-B, exemplary graphics 1100, 1105 are provided which indicate the frequency splitting and frequency shifting effects of the preamplifier designed according to the exemplary embodiments of the present invention. When two coils are positioned next to each other, and without any overlap, the coupling can cause a frequency splitting as shown in the graph 1100 of FIG. 11A. However, if a port of one coil is shorted as if, e.g., an ideal zero impedance preamplifier is connected, the frequency splitting effect of the equivalent circuit of FIG. 4 can be substantially reduced, and the resonance frequency of the circuit may shift to about 48.525 MHz, as shown in the graph 1105 of FIG. 11B. Such frequency shift may be predicted by the exemplary model of Equations (1)-(5) described hereinabove. In addition to the frequency shift, the Q factor of the circuit can also be reduced to about 203, thus resulting in the loading factor being reduced to about 5. This also facilitates a good performance in the exemplary circuit of the present invention.

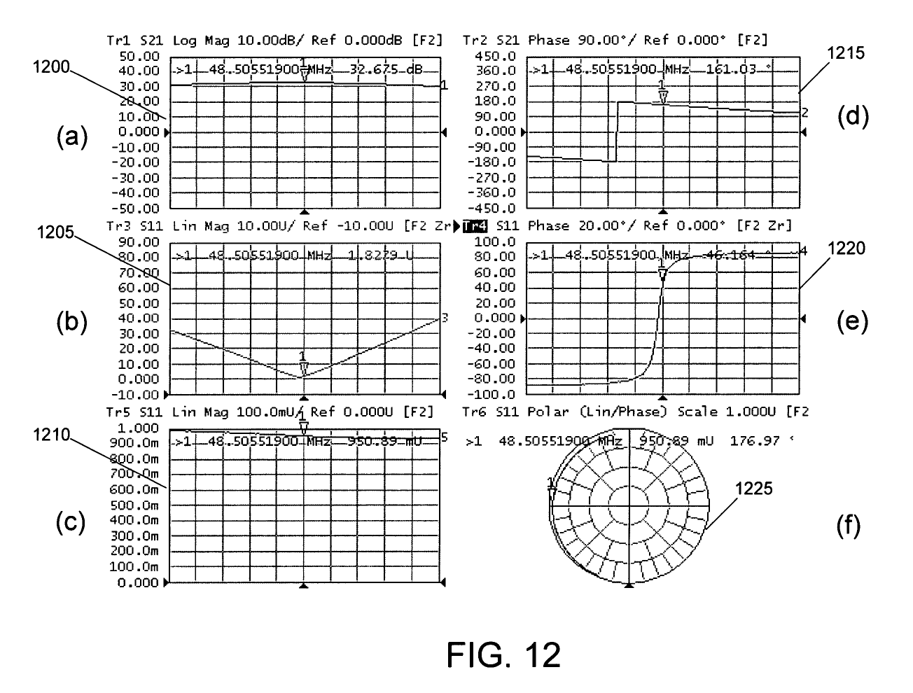

Preamplifier measurements may be conducted by using 24 units of 1T proton preamplifiers at 42.6 MHz, and retuning to 48.5 MHz. Referring to FIGS. 12A-F, exemplary graphs are provided which indicate the S-parameters of twenty four units of 1T proton preamplifiers according to the principles of the present invention. The graphs 1200, 1205 in FIGS. 12A-B indicate the magnitude and phase of S21 (e.g., 30 db˜32 db). For example, the graphs 1210, 1215 provided in FIGS. 12C-D show the magnitude and the phase of the input impedance (e.g., 0.8 ohm˜2.4 ohms), the graph 1220 provided in FIG. 12E indicates the magnitude of S11 on a linear scale, and the graph 1225 in FIG. 12F indicates the S11 parameter in polar coordinates, indicating that the phases of S11 are, e.g., each approximately 180°.

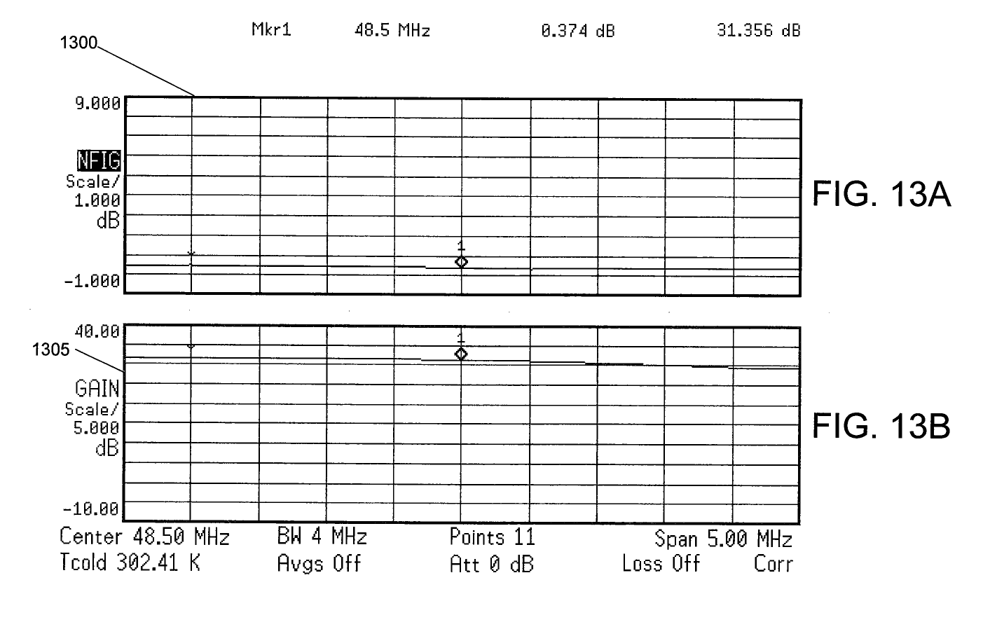

Referring to FIGS. 13A and 13B, exemplary graphs indicating the noise figures and gains of the preamplifiers built according to the exemplary embodiments of the present invention are provided. The gains and noise figures of the 24 units of 1T proton preamplifiers may be measured by a noise figure analyzer, such as Agilent N8973A, sold by Agilent Technologies Inc., of Palo Alto, Calif. An exemplary measurement can be seen in FIGS. 13A-B. The noise measurements of all preamplifiers can be about 0.4 dB. The gains of all preamplifiers may be about 31 dB. The gain and S21 parameter can be slightly different in this case due to the mismatch at the input of the preamplifiers.

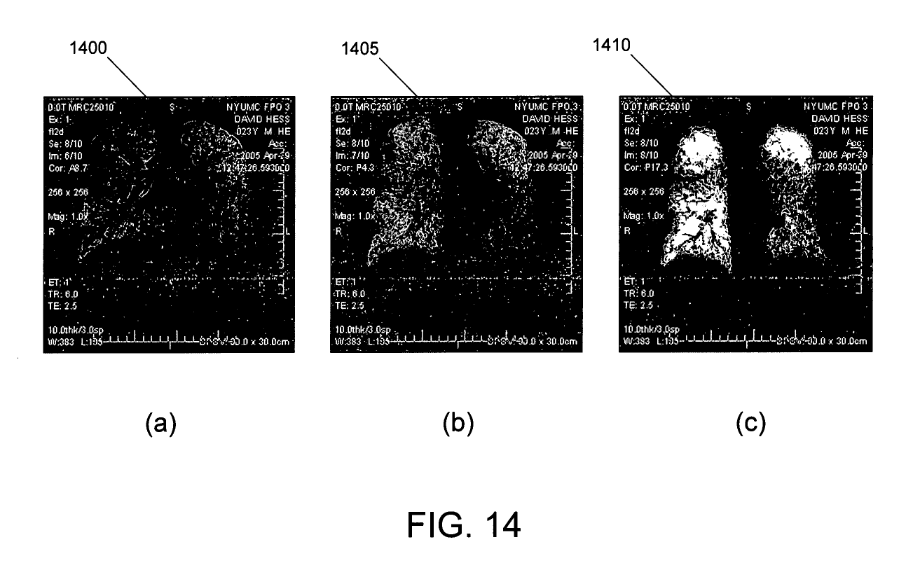

Referring to FIGS. 14A-14C, exemplary images acquired with an exemplary embodiment of the phased-array coil of the present invention are provided. Certain images 1400-1410 provided in FIGS. 14A-14C are lung images acquired in vivo using the exemplary phased-array coil of the present invention. Both 2D and 3D data acquisition capabilities may be examined. The images 1400-1410 provide three slices in a 3D parallel imaging data acquisition, where the following exemplary parameters were used: TR=6 ms, TE=2.4 ms, the data acquisition matrix was a 256×256 matrix, and the reduction factor iPAT was 8 (4×2).

As described herein above, a 24-element ³He exemplary phased-array apparatus can be provided in accordance with the exemplary embodiments of the present invention. A performance of this exemplary system indicates the feasibility of applying parallel imaging MRI with hyperpolarized ³He. The exemplary phased-array coil according to the exemplary embodiment of the present invention can significantly reduce the scan acquisition time, and improve the monitoring of a dynamic pulmonary function.

Other gases (such as ¹²⁹Xe, oxygen, or any other gases that may act as a contrast agent or otherwise aid in imaging) can be used in accordance with the present invention. In addition, certain materials that can be polarized and/or hyperpolarized and which may be provided in the form of a solid or liquid can be used as contrast agents in accordance with further exemplary embodiments of the present invention. For example, carbon-13 may be used as the contrast agent, and may be supplied in a solid form (e.g., a powder) and/or ingested. Alternatively, such exemplary contrast agent may be suspended in a liquid and injected into the body. The liquid that is capable of being polarized and/or hyperpolarized or which contains a suspended material that can be polarized and/or hyperpolarized may also be used. A contrast agent in liquid form may include, e.g., a liquid solvent and a polarizable or hyperpolarizable material contained in the solvent. Such contrast agent may be provided, e.g., as an emulsion, a suspension, or a solution. Such a liquid may be vaporized and inhaled, or it may be ingested or injected into the body.

A coil system that may be used in accordance with certain exemplary embodiments of the present invention can be, e.g., a rigid coil system, and/or it may include a 24-channel phased-array for the receiver and a 2-channel large loop for the transmitter. The exemplary coil system can include various numbers of elements, e.g., 128 elements, 256 elements, etc. The exemplary coil system can be provided in various configurations. Such exemplary configurations can include, e.g., a transmit/receive array, a receive only array, a loop array, or any suitable phase array configuration. For example, elliptical, cylindrical or planar strip arrays may also be used. The exemplary coil system can be tuned to a frequency appropriate for the particular hyperpolarized material or contrast agent used in a specific imaging procedure performed in accordance with certain exemplary embodiments of the present invention.

The foregoing descriptions of specific embodiments and best mode of the present invention have been presented for purposes of illustration and description only. They are not intended to be exhaustive or to limit the invention to the precise forms disclosed. Specific features of the invention are shown in some drawings and not in others, for purposes of convenience only, and any feature may be combined with other features in accordance with the principles of the invention described herein. Steps of the described processes may be reordered or combined, and other steps may be included. The embodiments were chosen and described in order to best explain the principles of the invention and its practical application, to thereby enable others skilled in the art to best utilize the invention and various embodiments with various modifications as are suited to the particular use contemplated. Further variations of the invention will be apparent to one skilled in the art in light of this disclosure and such variations are intended to fall within the scope of the appended claims and their equivalents. The publications referenced above are incorporated herein by reference in their entireties.

What is claimed is:

1. A system for performing parallel magnetic resonance imaging (“MRI”) using a contrast agent which is at least one of polarizable or hyperpolarizable, comprising:
a phased-array coil arrangement to be used in conjunction with the contrast agent; and
an amplification arrangement connected to the phased-array coil arrangement.

2. The system of claim 1, wherein the phased-array coil arrangement comprises a receive coil arrangement which further comprises a plurality of channels.

3. The apparatus of claim 2, wherein the phased-array coil arrangement comprises a transmit coil arrangement which further comprises a plurality of loops.

4. The apparatus of claim 3, wherein the phased-array coil arrangement comprises at least two shells.

5. The apparatus of claim 4, wherein each of the shells comprises an inner level portion and an outer level portion.

6. The apparatus of claim 5, wherein the receive coil is provided in the inner level of the shells.

7. The apparatus of claim 5, wherein the transmit coil is provided in the outer level of the shells.

8. The apparatus of claim 1, wherein the phased-array coil arrangement comprises a plurality of coils, and wherein at least two of the of adjacent coils overlap.

9. The apparatus of claim 1, wherein the phased-array coil arrangement comprises a plurality of coils, and wherein at least two of the of adjacent coils do not overlap.

10. The apparatus of claim 1, wherein the amplification arrangement comprises a low-impedance preamplifier arrangement that is tuned to a resonance frequency of the contrast agent.

11. The apparatus of claim 1, further comprising a frequency synthesis arrangement capable of connecting an MRI scanner with the phased-array coil arrangement and the amplification arrangement.

12. The apparatus of claim 1, wherein the phased-array coil arrangement comprises at least one of 128 elements or 256 elements.

13. The apparatus of claim 1, wherein the phased-array coil arrangement comprises at least one of a transmit/receive array, a receive-only array, or a loop array.

14. The apparatus of claim 1, wherein the phased-array coil arrangement has a form of at least one of an elliptical strip array, a planar strip array, or a cylindrical strip array.

15. The apparatus of claim 1, wherein the amplification arrangement is a low-impedance amplification arrangement.

16. The apparatus of claim 1, wherein the contrast agent has a form of a gas.

17. The apparatus of claim 16, wherein the gas comprises at least one of ³He or ¹²⁹Xe.

18. The apparatus of claim 1, wherein the contrast agent has a form of at least one of a liquid, an emulsion, a suspension, or a solution.

19. The apparatus of claim 1, wherein the contrast agent has a form of a solid material.

20. A method for performing parallel magnetic resonance imaging (“MRI”) using a contrast agent which is at least one of polarizable or hyperpolarizable, comprising:
introducing the contrast agent into a sample; and
acquiring images of at least one portion of the sample at least partially contacting the contrast agent using the parallel MRI

21. The method of claim 20 wherein the acquiring step is performed using a phased-array coil.

22. The method of claim 21, further comprising connecting a low-impedance preamplifier with the phased-array coil to perform the acquiring step.

23. The method of claim 22, further comprising connecting a frequency synthesis arrangement to an MRI scanner so as to connect the MRI scanner to the phased-array rigid coil and the low impedance preamplifier to perform the acquiring step.

24. A system for performing a parallel MRI procedure using a contrast agent which is at least one of polarizable or hyperpolarizable, comprising:
a first arrangement capable of generating at least one magnetic signal; and
a second arrangement adapted to receive at least two further signals which are associated with the at least one magnetic signal, wherein the second arrangement is capable of providing information associated with the parallel MRI procedure as a function of the at least two further signals.

25. The system of claim 24, wherein the first arrangement is capable of generating at least two of the at least one magnetic signals so as to facilitate the parallel MRI procedure using the contrast agent.

26. The system of claim 24, wherein the second arrangement is configured to perform the parallel MRI procedure using the contrast agent.

27. The system of claim 24, wherein the first arrangement comprises a phased array coil arrangement.
